(19) United States
(12) Patent Application Publication
(10) Pub. No.: US 2026/0102193 A1
(43) Pub. Date: Apr. 16, 2026

(54) DEVICES, SYSTEMS, AND METHODS FOR PULSED ELECTRIC FIELD TREATMENT OF TISSUE

(71) Applicant: Endogenex, Inc., Plymouth, MN (US)

(72) Inventors: Robert Anthony D'AGOSTINO, St. Louis Park, MN (US); Zackary John BECKER, St. Paul, MN (US)

(21) Appl. No.: 19/356,317

(22) Filed: Oct. 13, 2025

Related U.S. Application Data
(60) Provisional application No. 63/707,018, filed on Oct. 14, 2024.
(60) Provisional application No. 63/864,743, filed on Aug. 15, 2025.

(51) Int. Cl. A61B 18/00; A61B 1/00; A61B 34/30; A61M 25/10

(52) U.S. Cl. CPC A61B 18/00; A61B 1/00082; A61B 34/30; A61M 25/1002; A61B 2018/0022; A61B 2018/00666; A61B 2018/0072; A61B 2018/00767; A61B 2018/00827; A61B 2018/00875; A61B 2018/00904; A61B 2018/00982; A61B 2034/301; A61M 2025/1004; A61M 2025/1084; A61M 2205/0266; A61M 2205/3344

(57) ABSTRACT

Described here are devices, systems, and methods for treating tissue. In some variations, a method of treating a target tissue may comprise advancing a tissue treatment system and a visualization device to the target tissue of a patient. The tissue treatment system may comprise a tissue treatment device comprising an elongate body such as an endoscope, an inflatable balloon including an electrode array. The balloon may comprise a non-tapered distal end, one or more pleats and releasable fasteners configured to ensure a controlled unfolding inflation sequence of the pleats to achieve an expanded configuration, and a controlled folding deflation sequence to achieve a collapsed configuration.

### CROSS-REFERENCE TO RELATED APPLICATIONS

This application claims priority to U.S. Provisional Application No. 63/864743, filed Aug. 15, 2025, and U.S. Provisional Application No. 63/707018, filed Oct. 14, 2024, the entire contents of each of which is incorporated herein in their entirety; and wherein the entire contents of the following application is hereby incorporated in its entirety: U.S. application Ser. No. 19/188,563, filed Apr. 24, 2025.

### TECHNICAL FIELD

Devices, systems, and methods herein relate to inflatable balloons configured to be translated through a lumen such as a blood vessel or other bodily lumen such as the gastrointestinal tract.

### BACKGROUND

Diabetes is a widespread condition, affecting millions worldwide. In the United States alone, over 20 million people are estimated to have the condition. Diabetes accounts for hundreds of billions of dollars annually in direct and indirect medical costs. Depending on the type (Type 1, Type 2, and the like), diabetes may be associated with one or more symptoms such as fatigue, blurred vision, and unexplained weight loss, and may further be associated with one or more complications such as hypoglycemia, hyperglycemia, ketoacidosis, neuropathy, and nephropathy.

The treatment of chronic diseases such as obesity and diabetes through duodenal resurfacing has been proposed. For example, removing the majority of the mucosal cells from the section of the large intestine nearest the stomach may allow a rejuvenated mucosal layer to be regenerated, thereby restoring healthy (non-diabetic) signaling. Conventional treatments that apply thermal energy to the duodenum risk excessively heating and thus damaging more layers of the duodenum (e.g., muscularis) than desired, and/or must compensate for this excessive thermal heating. Conversely, conventional solutions may generate incomplete and/or uneven treatment. As such, additional systems, devices, and methods for treatment of duodenal, or other gastrointestinal tissue (“GI”) may be desirable.

In addition, angioplasty balloons are used in various ways to aid in opening occluded blood vessels. The balloons described herein without electrodes or electrode arrays may be useful in these procedures.

### SUMMARY

In some variations, the balloons described herein may be affixed to, and/or surround at least a portion of an endoscope.

In some variations, the balloons described herein may be affixed to, and or surround at least a portion of an elongate member, wherein one variation of an elongate member is a catheter.

In some variation, a treatment device comprising a balloon affixed to an elongate member such as a catheter, or to an endoscope comprising a 1:1 translation and torque of the elongate member or endoscope, and the balloon.

In some variations, the balloons described herein may be affixed or associated with an elongate member such as a catheter for use in, inter alia, angioplasty or other intravascular interventional procedures.

In some variations, fixation of the balloon to the elongate member or endoscope may comprise adhesive, tape, friction fit, interlocking features, heat sealing, laser welding, suction, and/or inflation of an intermediate member.

In some variations, the balloons described herein may be used as, or with, devices, systems, and methods for applying pulsed or modulated electric fields to tissue. These systems, devices, and methods may, for example, treat duodenal tissue of a patient to treat diabetes. In some variations, a system for treating tissue may comprise an elongate body, and a balloon coupled to the elongate body. The balloon may comprise one or more electrode arrays.

In some variations, the balloons described herein may be used to treat duodenal tissue or other GI tissue and may be affixed to a distal end of an endoscope.

In some variations, the balloons described herein may comprise one or more of a thermoplastic urethane, thermoplastic elastomer, polyethylene terephthalate, polyimide, nylon, and biaxially-oriented polyethylene terephthalate. In some variations, portions of the balloon may be substantially transparent or clear. In some variations, portions of the balloon may be opaque.

In some variations, the electrode array may comprise a flexible circuit substrate, wherein the flexible circuit substrate comprises one or more of the group consisting of: all-Polyimide laminate, Polyester (PET), Polyethylene Naphthalate (PEN), Polyamide, Liquid Crystal Polymer (LCP), and PTFE.

In some variations, the balloon may comprise a asymmetric portion, a cylindrical portion distal of the asymmetric portion, and a distal flat or semicircular end portion that is substantially transverse to a central axis of the cylindrical portion and/or an elongate member or endoscope when inflated or deployed. In some variations, when inflated the distal end may be everted beyond a distal end of the first asymmetric portion. In some variations, the distal portion may, when inflated, be inverted relative to a distal end of the first asymmetric portion.

In some variations, the balloon may comprise a symmetric portion, a cylindrical portion distal of the symmetric portion which is adjacent to a distal flat or semicircular end portion that is substantially transverse to a central axis of the cylindrical portion and/or an elongate member or endoscope when inflated or deployed. In some variations, when inflated, the distal end may be everted beyond a distal end of the symmetric portion. In some variations, the distal portion may, when inflated, be inverted relative to a distal end of the first asymmetric portion.

In some variations, a distal end of the balloon is not tapered.

The balloon of the present disclosure may comprise any therapeutically effective shape in combination with electrodes. For example, and without limitation, the balloon may be tapered at both proximal and distal ends, and cylindrical in the middle.

In some variations comprising an electrode array, the non-tapered distal end of some of the balloons described herein and/or the flat or semicircular or non-tapered, or circular distal end of the balloon allows precise location of the electrodes disposed on and/or around the balloon. For example, the electrodes, or electrode array or electrode sections attached to the cylindrical portion adjacent to the distal end of the balloon allows positioning of the electrodes at a distal tip of the endoscope. This allows precise location of the distal end of the electrodes which may coincide with the distal end of the balloon, i.e., the distal end of the larger diameter cylindrical portion, and/or the distal end of an endoscope and/or overtube during a GI procedure.

In some variations, when no electrodes are included, the balloon may be placed at a distal end of an elongate member such as an endoscope or catheter to allow for precise location of the distal end of the balloon, i.e., the distal end of the larger diameter cylindrical portion when executing a GI procedure, or an intravascular procedure such as angioplasty.

In some variations, a combined diameter of the system in the delivery configuration may be less than about 25 mm, or 20 mm, or 17 mm. In some variations, the balloon may comprise a proximal tapered portion. In some variations, the proximal tapered portion and the longitudinal axis form an angle between about 10 degrees and about 80 degrees. In some variations, the proximal tapered portion may be symmetric relative to the elongate member or endoscope. In some variations, the proximal tapered portion may be asymmetric relative to the elongate member or endoscope. In some variations, a portion of the asymmetric portion may be aligned with the elongate member or endoscope when inflated or deployed.

In some variations, the balloon may be eccentrically coupled to the elongate body such that a longitudinal axis of the elongate body does not align with the longitudinal axis of the balloon. In some variations, the elongate body may be coupled to a sidewall of the balloon. In some variations, the proximal tapered portion may comprise a first lateral taper and a second lateral taper that is asymmetric to and/or not aligned with the first lateral taper.

In some variations, the first lateral taper and the second lateral taper may be symmetric relative to each other.

In some variations, the balloon may be symmetric.

In some variations, the balloon may be asymmetric.

In some variations, the balloon may be eccentrically surround a portion of an elongate member such as an endoscope or a catheter.

In some variations, the balloon may be concentrically, or symmetrically surround a portion of an elongate member such as an endoscope or a catheter.

In some variations, the balloon's distal end comprises an aperture or tube in fluid communication with the interior of the balloon and configured to receive and seal around an elongate member such as a catheter or endoscope.

In some variations, the aperture or tube is everted or extends distally away from the remainder of the distal end. In some variations, the aperture or tube is inverted or extends proximally inward from the distal end. In some variations, an aperture is formed or defined through the distal end without inversion or eversion.

In some variations, the balloon is pleated to aid in folding to achieve a collapsed configuration.

In some variations, the pleated balloon may comprise stiffener member(s) or material(s) to ensure a desired, predetermined order of pleat opening and deployment during inflation, and a desired, predetermined order of pleat folding and collapse during deflation.

In some variations, the stiffener member(s) or material(s) may comprise a non-populated flex circuit material, PET, a metal such as stainless steel, a shape memory allow such as Nitinol, a shape memory polymer and/or a thin sheet or sheets, or coil(s) to stiffen portions of the pleat.

In some variations, the balloon may comprise one or more pleats formed of composite materials to help ensure pleat deployment order during pleat collapse or folding and opening during inflation.

In some variations, the material(s) forming the one or more pleats may be configured to provide an ordered deployment of the one or more pleats by requiring successively more inflation pressure to deploy each of the one or more pleats.

In some variations, the material(s) forming the one or more pleats may require an ordered folding of the one or more pleats during deflation.

In some variations, the material(s) forming the one or more pleats may aid in the balloon achieving a desired configuration or shape when inflated, and/or when deflated.

In some variations, the balloon comprises a plurality of fastener pairs that become unfastened to allow pleats to unfold and expand during an inflation or deployment procedure. In some variations the fastener pairs refasten during a collapsing of the balloon during deflation to aid in reaching a secured collapsed configuration. In some variations, the fastener pairs are of equal strength to enable the pleats to unfold at substantially the same time. In some variations, the fastener pairs are of unequal strength to enable a controlled expansion sequence for the pleats.

In some variations, the fasteners comprise magnet pairs. In some variations, the magnet pairs are of substantially equal strength. In some variations, the magnet pairs are of unequal strength. In some variations, three magnets may be associated with each other to provide two magnet pairs, with one magnet in common with the two magnet pairs.

In some variations, the magnet pairs are disposed on opposing sides or pleated regions of a central pleat.

In some variations, more than one pair of opposing magnets may be located along opposing sides or pleated regions adjacent to a pleat.

In some variations, a first pair of opposing magnets may be longitudinally spaced from a second pair of opposing magnets along opposing sides or pleated regions adjacent to a pleat.

In some variations, an overtube may be attached to the elongate member or endoscope and configured such that the inflatable balloon is affixed to and/or surrounds at least part of the overtube. In some variations, the length of the overtube may be less than the length of the elongate member or endoscope. In some variations, the length of the overtube may be equal to, or slightly longer than, the length of the balloon.

In some variations, the balloon is affixed directly to the elongate member or endoscope.

In some variations, the overtube may comprise a distal end feature that creates a positive engagement with a distal end region of the elongate member or endoscope.

In some variations, the endoscope may comprise a steerable tip such that the combination of endoscope and overtube enables full articulation of the endoscope.

In some variations, a system comprising the treatment device may further comprise the elongate member, or endoscope fixed in 1:1 translational and rotational position relative to the balloon.

In some variations, a treatment device is provided comprising an elongate member such as a catheter, or an endoscope and a balloon as described herein coupled to the elongate member, or endoscope. The balloon may comprise at least one pleat, and associated pleated opposing regions adjacent to the at least one pleat and configured to facilitate flattening of the balloon for placement into a delivery configuration. A width of the pleat and/or opposing pleated regions in the delivery configuration is about 0.1 mm to about the difference between a width of the balloon in the delivery configuration and a diameter of the elongate body.

In some variations, at least one pleat may comprise a first pleat and opposing sides or pleated regions adjacent to the first pleat on a first side of the balloon and a second pleat and opposing sides or pleated regions adjacent to the second pleat radially spaced apart from the first pleat. In some variations, the treatment device may comprise an electrode array configured to treat the tissue. The electrode array may be spaced apart from one or more of a proximal end and a distal end of the balloon by at least about 0.25 inches. In some variations, an electrode array may be coupled to the balloon. In some variations, at least one pleat and opposing sides or pleated regions adjacent to the at least one pleat may be configured to transition the balloon between a folded configuration having a first diameter and an unfolded configuration having a second diameter greater than the first diameter. In some variations, a lateral portion of the balloon may comprise at least one pleat and opposing sides or pleated regions adjacent to the at least one pleat. In some variations, the balloon may comprise one or more of a thermoplastic urethane, thermoplastic elastomer, polyethylene terephthalate, polyimide, nylon, and biaxially-oriented polyethylene terephthalate.

Also described herein is a treatment device comprising an elongate body and a balloon coupled to the elongate member or endoscope. The balloon may comprise an electrode array and a plurality of pleats configured to facilitate flattening of the balloon in an unexpanded configuration. When transitioning the balloon to an expanded configuration, a portion comprising the electrode array may expand before the plurality of pleats unfold.

In some variations, each of the plurality of pleats may fold radially inwards in the unexpanded configuration. In some variations, the portion of the balloon comprising the electrode array may comprise a first rigidity and one or more other portions of the balloon comprise a second rigidity different than the first rigidity. In some variations, a width of each pleat and opposing sides or pleated regions adjacent to each pleat may be up about the difference between a width of the electrode array and a diameter of the elongate body.

Also described herein is a treatment device comprising an elongate body, a balloon coupled to the elongate body, and an electrode array coupled to the balloon where the electrode array may comprise a substrate defining a plurality of apertures. A bonding layer may be bonded to the electrode array and the balloon using the apertures of the substrate.

In some variations, one or more, or a plurality, of pleats are provided with one or more releasable fasteners, for example a pair of two fasteners, in spaced-apart and aligned positions on and opposing sides or pleated regions adjacent to each pleat. In some variations, the releasable fasteners may comprise magnets. In some variations, the releasable fasteners may comprise opposing attractive magnets, a hook, loop or pin system, biocompatible sutures or fibers configured to break at a designated pressure, an adhesive, a heat-sensitive polymer with controlled degradation at body temperature, a shape-memory alloy such as nitinol with controlled changing of shape at body temperature.

In some variations, the releasable fasteners may be configured to provide for a controlled expansion of the pleats. In some variations, all pleats may be configured to inflate substantially simultaneously, whereby the releasable fasteners are all configured to release at substantially the same applied pressure within the exemplary inflatable balloon.

In some variations, the pleats and associated opposing sides or pleated regions adjacent to the pleats may be configured to begin inflating, or release from the securing releasable fasteners, at different internal applied balloon pressures during inflation. This variation may comprise, therefore, releasable fasteners configured to release at different, and controlled, internal applied balloon pressures. This configuration may allow a systematic inflation of pleats and opposing sides or pleated regions adjacent to the pleats in a predetermined order.

In some variations, inflated pleats opposing sides or pleated regions adjacent to the pleats may be configured to deflate in a controlled manner such that the releasable fasteners reengage to close and reform the pleat opposing sides or pleated regions adjacent to the pleat. In some variations, the closure or reformation of the pleats during controlled deflation may comprise a predetermined order of closure or reformation of the pleats opposing sides or pleated regions adjacent to the pleats.

In some variations, stiffened, or stiffening, member(s) may be used on, or along, the balloon to create a composite pleat structure that ensures intended pleat deployment order during inflation and deflation. In some variations, the stiffened members are more stiff than the surrounding balloon and/or balloon with electrode array. In some variations, the stiffening members may comprise a non-populated flex circuit material, stainless steel, a shape memory allow such as Nitinol, a shape memory polymer, PET, thin sheets, coils and the like.

In some variations, the stiffening member may bias the pleat structure to expand.

In some variations, the stiffening member may bias the pleat structure to collapse.

In some variations, a separate tube may be in fluid communication with the inflatable balloon and an inflation media reservoir and pump to inflate and deflate the balloon.

In some variations, the endoscope may comprise an insufflation and suction capability to inflate and deflate the balloon.

In some variations, an apposition test may be executed to determine appropriate luminal wall opposition. In some embodiments, the apposition test may comprise monitoring balloon pressure.

In some variations, electrodes may be tested or checked to confirm that the electrodes disposed on an inflated balloon are in sufficient contact with luminal tissue. In some variations, impedance may be measured to determine whether the electrode(s) is in sufficient contact with luminal tissue. In some variations, current may be measured to determine whether the electrode(s) is in sufficient contact with luminal tissue.

In some variations, current may be tested and checked to confirm that the electrodes are in sufficient contact with luminal tissue.

In some variations, a sub-therapeutic pulse, or pulses, may be generated and the resultant current measured to determine whether the electrode(s) is in sufficient contact with luminal tissue.

In some variations, current and/or impedance may be measured during a procedure and used to determine progress and/or whether the procedure is sufficiently complete.

In some variations, a treatment device comprising a balloon as described herein and an array of electrodes disposed thereon may execute a first treatment after inflating the balloon at a first treatment region, then after deflating at least slightly and translating the balloon to a second treatment region, executing a second treatment. In some variations, the first treatment and the second treatment overlap such that the overlap receives two treatments.

In some variations, the electrode array does not completely surround the inflatable balloon.

In some variations, the electrode array does completely surround the inflatable balloon.

In some variations, a second treatment may comprise rotating the balloon, elongate member or body, or the endoscope to treat tissue that was not treated with a first treatment.

In some variations, a treatment executed by a treatment device comprising an elongate member, or an endoscope with balloon affixed thereto, is executed by a controller. In some variations, the controller comprises a memory, processor and programmed instructions configured to instruct execution of the treatment. In some variations, the controller is configured to inflate and deflate the balloon. In some variations, the controller is configured to automatically inflate the balloon at a first treatment region, execute a first treatment, and, in some variations, at least partially deflate the balloon for translation to a second treatment region. In other variations, the controller is configured to execute a single treatment, then deflate for repositioning. In some variations, the controller is configured to automatically execute a status check to determine proper balloon deployment and/or sufficient electrode contact with tissue as described supra.

In some variations, a treatment executed by a treatment device comprising an elongate member, or an endoscope with balloon affixed thereto, is executed by a robotic system. In some variations, the robotic system comprises a robotic controller having a memory, processor and programmed instructions configured to instruct the robotic system to execute the treatment. In some variations, the robotic system comprises a robotic base including a drive unit in communication with the robotic controller and configured to manipulate an effector arm comprising a grasper configured to grasp the elongate member or endoscope. In some variations, the robotic system is configured to inflate and deflate the balloon. In some variations, the robotic system is configured to automatically inflate the balloon at a first treatment region, execute a first treatment, and in some variations: at least partially deflate the balloon, translate the balloon to a second treatment region, wherein the second treatment region overlaps the first treatment region, inflate the balloon and execute a second treatment. In other variations, the robotic controller is configured to execute a single treatment, then deflate for withdrawal. In some variations, the robotic system is configured to automatically execute a status check to determine proper balloon deployment and/or sufficient electrode contact with tissue as described supra.

### BRIEF DESCRIPTION OF THE DRAWINGS

FIG. 1 illustrates a schematic view of an illustrative variation of the present disclosure.

FIG. 2A illustrates a cross-sectional view of an illustrative variation of the present disclosure.

FIG. 2B illustrates a cross-sectional view of an illustrative variation of the present disclosure.

FIG. 2C illustrates a cross-sectional view of an illustrative variation of the present disclosure.

FIG. 2D illustrates a cross-sectional view of an illustrative variation of the present disclosure.

FIG. 2E illustrates a perspective cutaway view of an illustrative variation of the present disclosure.

FIG. 2F illustrates a perspective cutaway view of an illustrative variation of the present disclosure.

FIG. 2G illustrates a perspective cutaway view of an illustrative variation of the present disclosure.

FIG. 3A illustrates a side, cutaway view of an illustrative variation of the present disclosure.

FIG. 3B illustrates an end view of an illustrative variation of the present disclosure.

FIG. 3C illustrates an end view of an illustrative variation of the present disclosure.

FIG. 3D illustrates a side view of an illustrative variation of the present disclosure.

FIG. 3E illustrates a side view of an illustrative variation of the present disclosure.

FIG. 3F illustrates a side view of an illustrative variation of the present disclosure.

FIG. 4 illustrates one embodiment of an illustrative variation of the present disclosure.

FIG. 5 illustrates a schematic of an illustrative variation of the present disclosure.

### DETAILED DESCRIPTION

Described herein are devices, systems, and methods for treating tissue to address a chronic disease, or aid in opening an occluded blood vessel. For example, a pulsed electric field (PEF) system may be configured to generate a therapeutic pulsed electric field having predetermined bipolar, high current, short duration, electric pulses and applied to any body cavity or lumen (e.g., organ, vasculature, vessel) of a patient. The PEF treatment described herein may increase cell permeability and induce a targeted, non-thermal cellular apoptosis and/or necrosis while preserving Extracellular Matrix (ECM) tissue scaffold, promoting rapid epithelial layer replacement that reestablishes a neuroendocrine cell population with minimal inflammation. In this manner, a depth of penetration may be controlled and smooth muscle cells may be preserved, thereby leaving surrounding tissue undamaged.

In some variations, devices, systems, and methods may include those for treating diabetes and/or obesity by treating tissue within the gastrointestinal tract (e.g., duodenal tissue) of a patient. In some variations, treatment of the duodenum may comprise treating at least about 30% of the mucosal lining of the duodenum with minimal trauma, and no permanent damage or scarring to the vasculature and muscles. For example, mucosal and/or submucosal cells of the duodenum may be treated using a pulsed electric field (PEF) system configured to generate a therapeutic pulsed electric field. The application of a pulsed electric field to duodenal tissue may affect individual parts or mechanisms within a cell (e.g., depth of tissue treated), that can be specifically targeted based on electrode geometry and the frequency, intensity, and duration of the pulses.

Systems described here may include one or more of the components used to treat tissue, such as, for example, a pulsed electric field device and a visualization device. Suitable examples of such systems and devices are described in International Application Serial No. PCT/US2022/025630, filed on Apr. 20, 2022, and U.S. Patent Application Ser. No. 63/563,149, filed on Mar. 8, 2024, U.S. patent application Ser. No. 19/885,563, filed on Apr. 24, 2025, U.S. Provisional Application Ser. No. 63/638851, filed Apr. 25, 2024, and U.S. Provisional Application Serial No. 63/707018, filed Oct. 14, 2024, the disclosure of each of which is hereby incorporated by reference in its entirety.

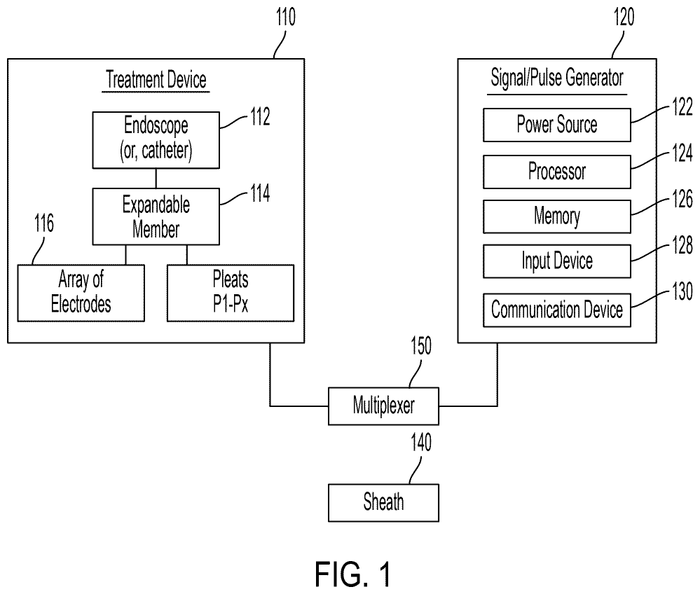

As shown in FIG. 1, an exemplary tissue treatment system 100, e.g., a PEF system, may comprise a treatment device 110 comprised of an endoscope 112 with an expandable member 114 such as an inflatable balloon affixed thereto and an array of electrodes 116 disposed on the inflatable balloon and wherein the balloon comprises one or more pleats P1-Px as described herein. In addition, a signal or pulse generator 120 is provided and comprising a power source 122, a processor 124 for executing programmed or programmable instructions stored within a memory 126, an input device 128 such as a keyboard, and a communication device 130.

In some variations, the communication device 130 may be configured to permit an operator to control one or more of the devices of the PEF system. The communication device may comprise a network interface configured to connect the tissue treatment device to another system (e.g., Internet, remote server, database) by wired or wireless connection. In some variations, the tissue treatment device may be in communication with other devices (e.g., cell phone, tablet, computer, smart watch, and the like) via one or more wired and/or wireless networks. In some variations, the network interface may comprise one or more of a radiofrequency receiver/transmitter, an optical (e.g., infrared) receiver/transmitter, and the like, configured to communicate with one or more devices and/or networks. The network interface may communicate by wires and/or wirelessly with one or more of the tissue treatment device, network, database, and server.

In some variations, the treatment device 110 may further comprise a handle (not shown, but as well understood in the art) connected with a proximal end of the exemplary endoscope 112, and one or more sensors. The system may further comprise a sheath 140 having a lumen through which the treatment device 110 may be translated and/or rotated.

In some variations, the tissue treatment device 110 may comprise a delivery configuration and a treatment configuration. The tissue treatment device 110 may be placed in the delivery configuration when advanced to a predetermined tissue, when repositioned within a body cavity or lumen, and/or when removed from the body. For example, in the delivery configuration, the balloon 114 may be deflated and positioned circumferentially about the endoscope 112 that may be within a sheath. The tissue treatment device 110 may transition between the delivery and treatment configurations when, for example, the device 110 is positioned at or near the predetermined tissue. For example, in the treatment configuration, at least a portion of the balloon 114 may be translated distal to the sheath 140, or the sheath 140 translated proximally to expose the balloon 114, to enable the balloon 114 to be inflated for treatment. In some variations, in the treatment configuration, the balloon 114 may be placed in (e.g., transition to) one or more of an unexpanded and flattened configuration, an expanded configuration, and a partially expanded configuration.

Generally, the signal generator 120 may be configured to provide energy (e.g., energy waveforms, pulse waveforms) to the tissue treatment device 110 to treat predetermined portions of tissue, such as, for example, duodenal tissue. In some variations, a PEF system 100 as described herein may include a signal generator 120 that comprises an energy or power source 122 and a processor 124. The signal generator 120 may be configured to deliver a bipolar waveform to an electrode array, which may deliver energy to the tissue (e.g., duodenal tissue). The delivered energy may aid in resurfacing, regenerating or otherwise treating the desired tissue and/or cells while minimizing damage to surrounding tissue. In variations in which the desired tissue is duodenal tissue, the delivered energy may aid in resurfacing the mucosa of the duodenum while minimizing damage to surrounding tissue (e.g., muscularis tissue). In some variations, the signal generator 120 may generate one or more bipolar waveforms.

In some variations, in order to limit nerve stimulation, a pulse waveform may, on average, comprise a net current of about zero (e.g., generally balanced positive and negative current), and have a non-zero time of less than about 2 μsec or less than about 5 μsec. In some variations, the pulse waveform may comprise a square or rectangular waveform. For example, the pulse waveform may comprise a square or rectangular shape in voltage drive and in current drive, or the pulse waveform may comprise a square or rectangular shape in voltage drive and a sawtooth shape in current drive. In some variations, the pulse waveform may comprise a sawtooth shape in voltage drive and/or voltage controlled mode. In some variations, one or more pulses may comprise a half sine-wave for both current and voltage. In some variations, one or more pulses may comprise different rise and fall times. In some variations, one or more pulses may comprise a bipolar pulse at a first potential followed by pulse pairs at a second potential less than the first potential.

In some variations, a multiplexer 150 may be coupled to the tissue treatment device as shown in FIG. 1. For example, the multiplexer 150 may be coupled between the signal generator 120 and the tissue treatment device 110, or the signal generator 120 may comprise the multiplexer. The multiplexer 150 may be configured to select a subset of electrodes of an electrode array 116 receiving a pulse waveform generated by the signal generator 120 according to a predetermined sequence. For example, in some variations, the electrode array 116 may comprise one or more sections that correspond to a subset of electrodes. The electrode array 116 may comprise between 1 and 10 sections, including 1, 2, 3, 4, 5, 6, 7, 8, 9, or 10 sections. Each section may comprise the same number of electrodes and/or the same surface area as every other section, but need not. The predetermined sequence may be optimized to treat tissue at a given treatment site. Additionally or alternatively, the multiplexer 150 may be coupled to a plurality of signal generators 120 and may be configured to select between a waveform generated by one of the plurality of signal generators 120 for a selected subset of electrodes.

In some variations, the multiplexer 150 and the signal generator 120 may be configured to deliver a pulsed electric field waveform to two or more non-proximate sections (e.g., first section, second section) of the plurality of sections in a predetermined sequence.

In some variations, the electric field may decay such that the electric field strength is less than about 400 V/cm at about 3 mm from the inner surface of the duodenum. In some variations, a predetermined bipolar current and voltage sequence may be applied to an electrode array of the pulsed electric field device to generate the pulsed or modulated electric field. The generated pulsed or modulated electric field may be substantially uniform to robustly induce cell death in a predetermined portion of duodenal tissue. For example, a generated pulsed or modulated electric field may spatially vary up to about 20% at a predetermined depth of tissue, between about 5% and about 20%, between about 10% and 20%, and between about 5% and about 15%, including all ranges and sub-values in-between. Furthermore, the pulsed electric field device may be biocompatible and resistant to stomach acids and intestinal fluids.

In some variations, a tissue treatment system, or PEF 100 may comprise a pulsed electric field device configured to treat tissue (e.g., regenerate mucosal and submucosal cells of the duodenum). The pulsed electric field device in a delivery configuration (e.g., a balloon of the pulsed electric field device) may be disposed in some variations within an optional sheath 140 and releasably coupled to a visualization device (e.g., endoscope) 112, such as, for example, to an outer surface of the visualization device 112. The pulsed electric field device 110 may comprise a balloon 114 such as an inflatable balloon having an electrode array 116 coupled thereto. The balloon 114 and the electrode array 116 may be advanced distal to the visualization device 112 and transitioned to a treatment configuration when the device 110 is located at a tissue treatment site. The balloon 114 may be positioned and inflated to create apposition between the electrode array 116 and to the tissue. The electrode array 116 may generate a pulsed electric field to treat the tissue.

In some variations, the balloon may not comprise electrodes and may be affixed to an elongate member such as a catheter and configured to perform interventional vascular procedures such as angioplasty.

### Balloon

Generally, the balloons and/or other expandable members such as stents or other mechanically expandable devices, described herein may be configured to transition between delivery and treatment configurations to facilitate delivery with a visualization device through a patient body and aid in positioning the electrode array relative to target tissue during a treatment procedure. For example, the balloon (e.g., inflatable member such as an inflatable balloon) may expand to contact tissue. In some variations, contact between the balloon and the tissue may hold, or assist in holding, the pulsed electric field device (e.g., elongate body, electrode array, sensor) in place relative to the tissue without catching or trapping tissue. As described above, the balloon may comprise an electrode array. The electrode array may be or otherwise comprise any of the electrode arrays described herein. In some variations, such as, for example, when the balloon comprises an exemplary inflatable balloon, the electrode array may be disposed on an outer surface of the balloon. In other variations, the electrode array may be disposed on an inner surface of the balloon. In other variations, the electrode array may be disposed within a wall of the balloon. In some variations, the balloon may comprise a lumen therethrough configured to receive the visualization device.

In addition to the balloons described in the references that are incorporated herein by reference, the following balloon designs may also be used to execute intralumenal procedures such as, without limitation, PEF, irreversible electroporation, and angioplasty or other intravascular interventional procedures.

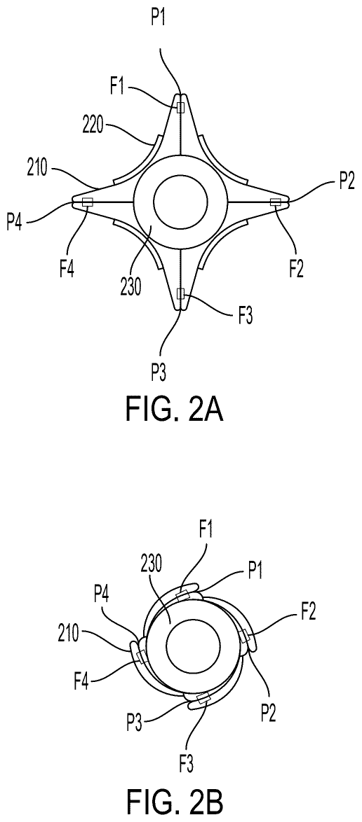

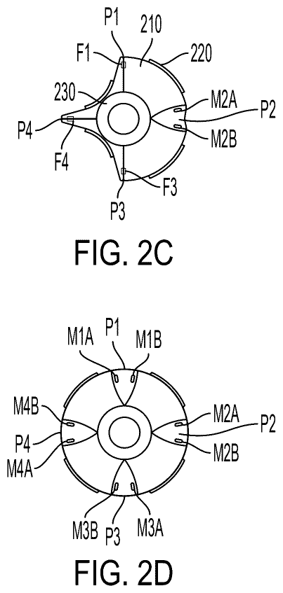

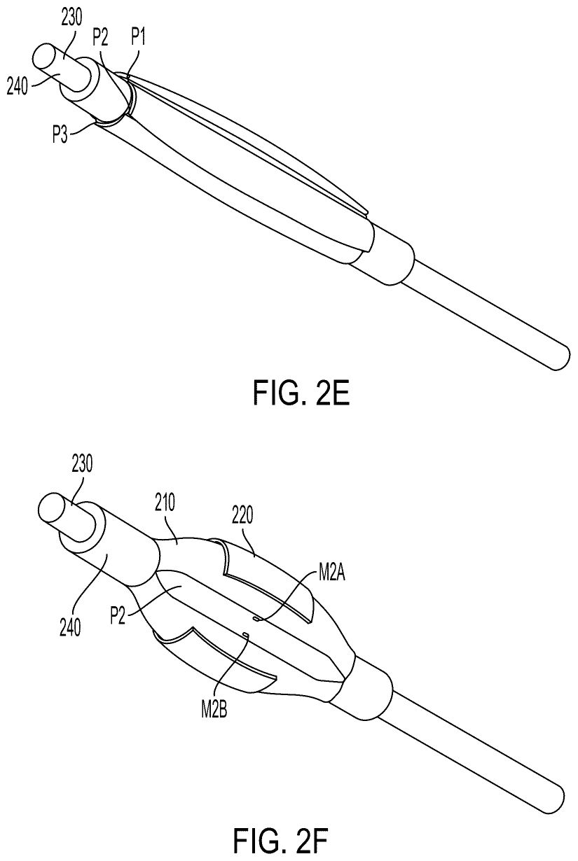

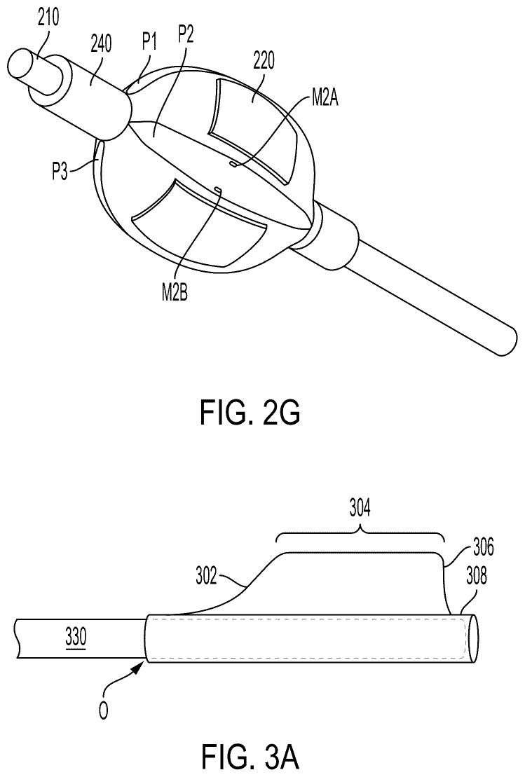

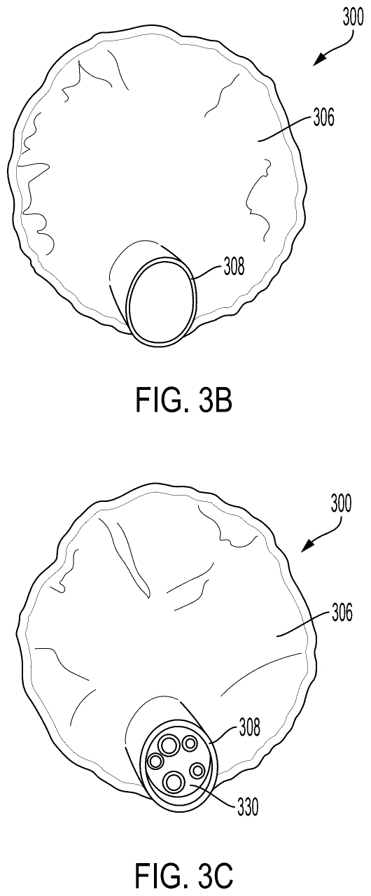

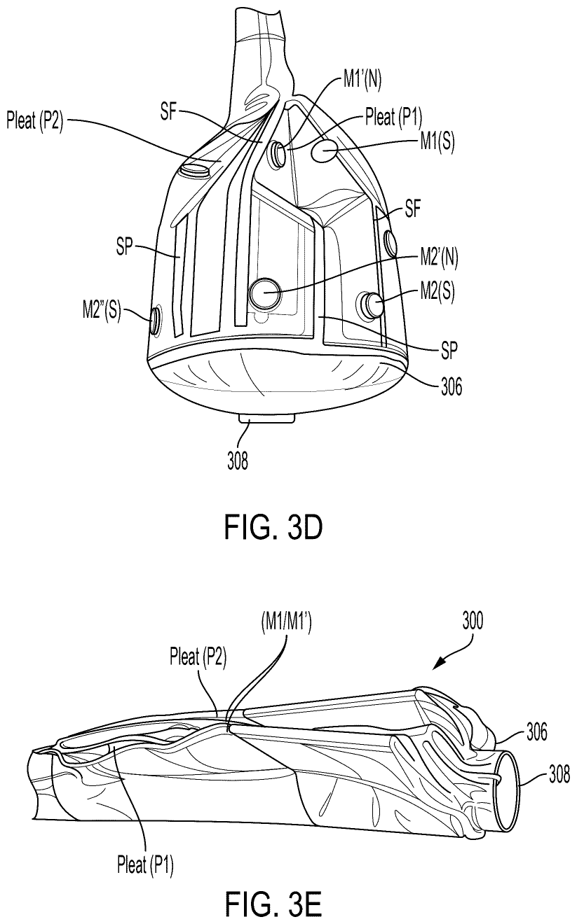

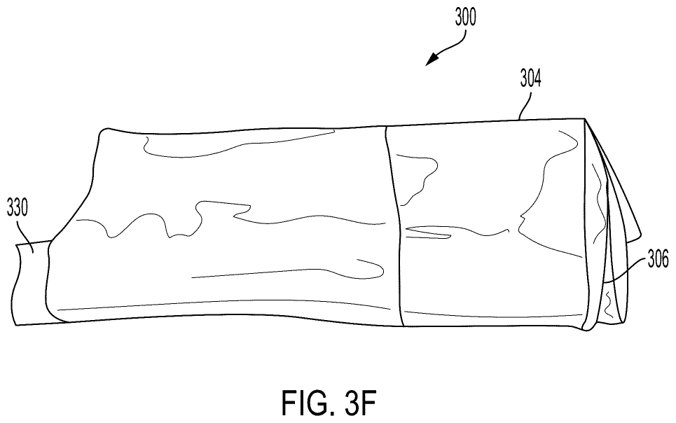

Turning now to FIGS. 2A-2G, devices and methods for securing and deploying or inflating an exemplary pleated balloon are provided. The exemplary balloon of FIGS. 2A-2G is symmetric when inflated. However, the concepts described herein for FIGS. 2A-2G that are related to pleats, and the predetermined ordering of deployment and folding of the pleats using releasable fastener pairs are fully applicable to the variations discussed and illustrated by FIGS. 3A-3F.

Known inflatable balloons comprise a plurality of pleats that are configured to achieve an unexpanded or delivery configuration that expand in a non-specified order as the inflation proceeds and the related pressure within the balloon increases. In addition, after deflation, known inflatable balloons simply collapse in an uncontrolled form without reformation of the pleats.

In some variations, one or more, or a plurality, of pleats are provided with two releasable fasteners in spaced-apart and aligned positions on each pleat. In some variations, the releasable fasteners may comprise magnets. In some variations, the releasable fasteners may comprise one or more of the group consisting of: opposing attractive magnets, a hook, loop or pin system, biocompatible sutures or fibers configured to break at a designated force or pressure, an adhesive, a heat-sensitive polymer with controlled degradation at body temperature, a shape-memory alloy such as nitinol, and shape-memory polymers.

In FIGS. 2A-2G, devices and methods for controlled expansion of pleated regions of an inflatable balloon 210 are illustrated. As used herein, pleated regions comprises a pleat, that is an area or line disposed to fold radially inward, and the adjacent regions on either side of the pleat that, when folded, are opposing surfaces that are at least partially in contact with each other.

Variations of the devices and methods may comprise controlled deflation of the pleated regions. Some variations allow for a predetermined sequence of expansion of pleated regions.

FIG. 2A illustrates in cross-section a balloon comprising an exemplary inflatable balloon 210 comprising 4 pleats, or pleated regions, P1, P2, P3, P4 in a deflated or closed configuration, wherein the pleats P1, P2, P3, P4 are shown as spaced-apart inversions of the balloon 210 material. The balloon 210 is secured to and surrounding, or partially surrounding, and endoscope 230. In some variations, the balloon 210 may form a distal tip comprising an aperture or tube 240 to which the endoscope 230 may be sealed. In some variations, the endoscope 230 may extend distally through the aperture or tube 240, while in other variations a distal end of the endoscope 230 may be positioned at a distal end of the aperture or tube 240.

In variations, an overtube (not shown) may be provided surrounding at least a distal end of the endoscope 230, to which the balloon 210 is secured to. The artisan will readily recognize that the variation of FIG. 2A is merely exemplary, showing 4 pleats, or pleated regions that are equally spaced apart around the endoscope 230. Other variations may comprise unequal spacing between the pleats or pleated regions. Other variations may comprise one, or more than one, pleat or pleated region. For example, 2 pleats or pleated regions, may be provided in some variations, wherein the pleats are spaced circumferentially apart by 180 degrees. Other variations may comprise balloons that are asymmetric when inflated. Exemplary electrode sections 220 are shown attached to the balloon 210. The electrode section variant of FIGS. 2A-2G illustrate discrete, spaced-apart electrode sections 220. In other variations, the electrode sections may be in contact so as to not create spacing between adjacent electrode sections. In other variations, two adjacent electrode sections may be spaced apart while the remaining adjacent electrodes sections are in contact with each other. Other variations may comprise the balloon of FIGS. 3A-3F discussed infra.

Further, as discussed above, the balloon 210 may comprise an asymmetric shape relative to the endoscope 230, such as described below and illustrated in FIGS. 3A-3F. In this variation, as above, one or more than one pleat or pleated region, and pair(s) of fasteners as discussed below, may be provided and functions as described herein.

Returning to FIGS. 2A-2G, four pairs of releasable fasteners F1, F2, F3, F3 are associated with each pleated region P1, P2, P3, P4. As noted above, releasable fasteners may in some variations, one or more, or a plurality, of pleats are provided with two releasable fasteners in spaced-apart and aligned positions on each pleat. In some variations, the releasable fasteners may comprise magnets. In some variations, the releasable fasteners may comprise one or more of the group consisting of: opposing attractive magnets, a hook, loop or pin system, biocompatible sutures or fibers or tether configured to break at a designated force or pressure, an adhesive, a heat-sensitive polymer with controlled degradation at body temperature, a shape-memory alloy such as nitinol, and shape-memory polymers.

Each of the illustrated pairs of releasable fasteners F1, F2, F3, F4 comprise a type of releasable fastener, exemplary pairs of opposing aligned and attracting magnets, M1A, M1B; M2A, M2B; M3A, M3B; and M4A, M4B. Each pair of opposing aligned and attracting magnets are configured to connect or engage in close proximity at the point at which the magnet pairs exert a maximum attractive force on each other. This results in a securement and closure of each pleat P1, P2, P3, P4 in preparation for wrapping or folding around the elongate body or overtube 230.

In some variations, one pleat, or two pleats, or more than two pleats may be provided. In some variations, at least one of the pleats may be provided without a releasable fastener while the remaining pleats may comprise a releasable fastener.

Closing or securing a pleated region P1, P2, P3, P4 comprises connecting the associated releasable fastener F1, F2, F3, F4 such that the material of each opposing pleated region adjacent exemplary balloon's 210 pleat is in touching or close association as in FIG. 2B. The associated releasable fastener F1, F2, F3, F4 ensures that the pleated region P1, P2 remains in the desired collapsed shape until the releasable fastener F1, F2, F3, F4 releases.

In general, the releasable fastener(s) may comprise one or more of: pairs of opposing attractive magnets, a hook, loop or pin system, biocompatible sutures or fibers configured to break at a designated force or pressure, an adhesive, a heat-sensitive polymer with controlled degradation at body temperature, a shape-memory alloy such as nitinol, and shape-memory polymers. Each of these structures are configured to connect with opposing sides of a pleat or pleated region to secure the opposing sides against each other, or nearly against each other. In other variations, the releasable fastener(s) are configured to prevent inflation expansion of the secured pleat or pleated region until a predetermined internal balloon pressure is generated. Once that predetermined pressure is generated in the balloon, the releasable fastener(s) are configured to release the pleat or pleated region to enable inflated expansion of same.

In the variation illustrated, wherein each one of the at least one releasable fastener comprises a pair of opposing magnets configured to attract each other, each opposing magnet produces a magnetic field, such that the opposing magnets are configured to exert an attraction force that increases as the distance between the opposing magnets decreases, reaching a maximum attractive force magnitude when the opposing magnets are in close engaged proximity to each other. In this manner, the opposing magnets, when exerting the maximum attractive force, result in the securement and closure of the associated pleat or pleated region, wherein the pleat(s) or pleated region(s) are deflated.

Inflation of the balloon, or inflatable balloon, generates an inflation pressure that produces a force on the releasable fastener(s), including the illustrated variation comprising opposing magnets. When the generated pressure produces a force that is greater than the ability of the releasable fastener(s) to remain engaged or intact, the releasable fastener will release the secured and closed pleat. In the variation of opposing magnets, the internal balloon pressure within the relevant pleat or pleated region will, when producing a force greater than the maximum attractive force, cause the magnets to move apart from each other. In all cases, after release of the releasable fastener(s), the associated pleated region will proceed to an inflated configuration.

In some variations, the pressure, and related force, required to induce the releasable fastener to release a pleat or pleated region may be the same for all pleats or pleated regions, and associated fastener(s) of an inflatable balloon. In some variations, the releasable fasteners may be configured to release at different internal balloon pressures, providing for a predetermined release, and inflation, sequence for the pleats or pleated regions.

Deflation of an inflated balloon with pleats or pleated regions comprising pairs of opposing attractive magnets as discussed above, may result in the opposing magnets of each pair moving closer again to each other such that the attractive force therebetween ultimately becomes the maximum attractive force, whereby the pairs of magnets again secure and close the pleats or pleated regions.

FIG. 2B illustrates the secured and closed pleats, or pleated regions P1, P2, P3, P4 in a wrapped or folded configuration around the endoscope 230. The configuration of FIG. 2B is shown in perspective view in FIG. 2E.

FIGS. 2C and 2D illustrate one exemplary variation of a controlled inflation and expansion of the pleats, or pleated regions P1, P2, P3, P4. In this example, pleat, or pleated region, P2 is the first pleat to comprise a release of releasable fastener F2, resulting in inflation and expansion of pleat P2. The pair of magnets M2A, M2B comprising releasable fastener F2 are configured to release at a generated internal balloon force during inflation that is a lower magnitude than the remaining releasable fasteners F1, F3, F4.

As shown in FIG. 2D, subsequent to the initial inflation and expansion of pleated region P2, releasable fasteners F1, F3 and F4 release, based on the internal force generated by the inflation of the inflatable balloon 210, either in series or at substantially the same time. This results in the inflation and expansion of pleats or pleated regions P1, P3 and P4. The configuration of FIG. 2C is shown in perspective view in FIG. 2F. The configuration of FIG. 2D is shown in perspective view in FIG. 2G. In some variations, a controlled sequence of pleat inflation may be achieved with a predetermined order of pleat inflation, and/or deflation. In other variations, the pleats may be inflated, and deflated, at substantially the same time.

As best seen in FIGS. 2C and 2D, the first magnet M1A of the releasable fastener F1 is spaced apart from the second magnet M1B of the releasable fastener pair F1 within the expanded and inflated pleat P1. Similarly, the first magnet M2A of the releasable fastener pair F2 is spaced apart from the second half M2B of the releasable fastener pair F2, the first magnet M3A of the releasable fastener pair F3 is spaced apart from the second magnet M3B of the releasable fastener pair F3, and the first magnet M4A of the releasable fastener pair F4 is spaced apart from the second magnet M3B of the releasable fastener pair F4. The first and second magnets, e.g., M1A, M1B; M2A, M2B; M3A, M3B, M4A, M4B of each of the releasable fastener pairs F1, F2, F3, F4, may be located on an inner surface of the associated pleat, encapsulate or encased or sealed within the associated pleat walls and/or on an outer surface of the associated pleat. In some variations, the releasable fastener pairs F1, F2, F3, F4 may be adhered to an inner and/or outer surface of the associated pleated region P1, P2, P3, P4.

As discussed above, in an exemplary variation, one or both of the releasable fastener pairs F1, F2, F3, F4 may comprise opposing attractive magnet pairs wherein the pleated regions P1, P2, P3, P4 may be manually closed, i.e., each magnetic half of the respective releasable fastener pairs F1, F2. F3, F4 manually moved closer to each other, until the attractive force of the opposing magnets within each pleated region P1, P2, P3, P4 engages to connect the opposing magnets, closing each of the pleated region P1, P2. P3, P4, wherein the maximum attractive force is at a maximum for each fastener pair F1, F2, F3, F4. The maximum attractive force may be substantially the same for each fastener pair F1, F2, F3, F4. In some variations, the maximum attractive force may be different for one or more of the fastener pairs. In an exemplary variation comprising opposing magnets to form the releasable fastener pairs, the maximum attractive force the magnet pair is capable of exerting occurs when the magnets are in the closest possible proximity to each other. The magnetic attractive force decreases exponentially as the distance between the magnets increases. In some variations, opposing magnets located along the pleat(s) are configured to attract each other, wherein each opposing magnet produces a magnetic field, and wherein the opposing magnets are configured to exert an attraction force comprising a magnitude when the opposing magnets' magnetic fields are overlapping.

In some variations, the releasable fastener pairs may be configured to provide for a controlled expansion of the pleats. In some variations, all pleats may be configured to inflate substantially simultaneously, whereby the releasable fasteners are all configured to release at substantially the same applied pressure within the exemplary inflatable balloon. In some variations, the releasable fasteners comprise a release pressure threshold, wherein the releasable fasteners remain closed until subjected to internal balloon pressure that is greater than the release pressure threshold. In the exemplary variation wherein the releasable fastener pairs comprise opposing, attractive, magnets, the magnetic force exerted when the magnets are in closest proximity to each other comprises the force that release pressure threshold must generate to pull the magnets apart from each other.

In some variations, the releasable fasteners may be configured to release at different release pressure thresholds during inflation such that the associated pleats expand and inflate at different times. This configuration may allow a controlled systematic inflation of pleats in a predetermined order. In other variations, the releasable fasteners may be configured to release at substantially the same release pressure threshold to allow a controlled inflation of the pleats at substantially the same time.

In some variations, inflated pleats may also be configured to deflate in a controlled manner such that the releasable fasteners reengage to close and reform the pleat. In some variations, the closure or reformation of the pleats during controlled deflation may comprise a predetermined order of closure or reformation of the pleats.

Some variations may comprise one pleat, while other variations may comprise more than one pleat. A preferred variation comprises two pleats, a first pleat and a second pleat. Some variations include pleat 1 comprising an electrode array along at least a portion of pleat 1. Some variations include pleat 2 without an electrode array 320. In some variations, pleat 1 does not comprise releasable fasteners. In some variations, pleat 2 does comprise at least one releasable fastener, e.g., at least one pair of opposing magnets, e.g., M1A, M1B as in FIG. 2A.

Because there may not be a releasable fastener associated with pleat 1, but there are releasable fastener(s) associated with pleat 2, greater inflation-generated force will be required to open pleat 2 compared with pleat 1. Thus, during inflation, pleat 1 inflates and expands first, followed by inflation and expansion of pleat 2 as the exemplary magnetic attractive force between the exemplary at least one pair of opposing magnets M1A, M1B is exceeded by the inflation-generated force.

During deflation, the opposite sequence may occur. Pleat 2 may collapse and close first due to the decreasing distance between the at least one pair of magnets M1A, M1B resulting in increasing magnetic attraction force therebetween until the maximum attraction force is generated, wherein the magnet pair M1A, M1B are in close engaged proximity with each other and pleat 2 is closed and resecured. As deflation continues, pleat 1 fully deflates and closes after pleat 2.

An analogous variation comprises a symmetric balloon as described above, with 2 exemplary pleats, wherein one pleat comprises at least one releasable fastener, e.g., opposing magnet pairs M1A, M1B, and the other pleat does not comprise a releasable fastener.

In these variations, pleat 2 may function to ensure that sufficient tissue contact or apposition is achieved after balloon inflation is executed.

Turning now to FIGS. 3A-3F, variations of an alternate balloon configuration 300 are shown.

Generally, the variations of FIGS. 3A-3F comprise a proximal tapered section 302, a distal section 304 that may in some variations be cylindrical and comprising a distal end 306. The distal section 304 may comprise the largest diameter of the balloon 300 when in an inflated configuration, and the distal end may be circular with a diameter in some variations the same as the diameter of the distal section when it is cylindrical. In some variations, the proximal end of the balloon 300 may also be circular, similar to the circular variation of distal end 306. As discussed herein, the middle section may comprise an electrode array 308 that may comprise electrode sections, such that inflation of the distal section 304 places the electrodes of the electrode array 306 and/or electrode sections in contact with the target tissue.

The distal end section 306 may be arranged generally transverse or perpendicular to a longitudinal axis A of an elongate member or endoscope. As shown, an endoscope 330 is provided, wherein the balloon 300 is affixed at a distal end region, or distal end of, the endoscope 330. In other variations, the distal end section 306 may be angled relative to a longitudinal axis A of the elongate member or endoscope 330. In some variations, the distal end section 306 may be substantially flat when the balloon 300 is inflated. In some variations, the distal end section 306 may form a semicircular shape when the balloon 300 is inflated. In some variations, the semicircular shape may form an everted profile wherein at least a portion of the everted flat distal end 306 extends distally beyond a distal end of the distal section 304. In some variations, the semicircular shape may form an inverted profile wherein at least a portion of the inverted flat end 306 extends proximal to a distal end of the distal section 304.

Known inflatable balloons that are configured to be deflated, collapsed and translated within a lumen, e.g., a blood vessel or gastrointestinal tract, comprise proximal and distal tapered ends, with a cylindrical middle portion therebetween. The variations of the balloons discussed presently provide a distal end 306 which effectively interrupts the large diameter middle section of the balloon by closing the balloon without requiring a distal taper, i.e., by providing a non-tapered distal end 306, wherein the non-tapered distal end 306 may be substantially orthogonal to a longitudinal axis of the elongate member or endoscope 330 as illustrated.

The distal end 306 of the inflatable balloon 300 may define a distal tube 308 configured to receive an elongate member such as a catheter, or an endoscope 330. The distal tube 308 may extend away distally from the distal end 306 of the balloon 300, thereby forming a short tube. In some variations, an inner surface of the distal tube 308 of the balloon may be configured to seal against an outer surface of the elongate member or endoscope 330. In some variations, an overtube O is provided and which is fixedly attached to at least a portion of the length of the elongate member or endoscope 330, preferably disposed at a distal region, e.g., a distal end region, of the endoscope 330. In some variations, the overtube O is provided such that an inner portion of the distal tube 308 is configured to seal against an outer portion of the overtube.

As illustrated in FIGS. 3A-3C, the distal tube 308 of the distal end 306 is offset radially to the edge of the distal end 306. Other variations may locate the distal tube 308 of the distal end 306 at any location within the distal end 306.

As shown in FIG. 3C, in some variations, a distal end of the elongate member or endoscope 330 may terminate at, or near, a distal end of the distal tube 308. In some variations, the distal end of the elongate member or endoscope 330 may be positioned within the distal tube 308 such that a distal end of the elongate member or endoscope 330 is proximal to the distal end of the distal tube 308. In some variations, the elongate member or endoscope 330 may extend distally beyond a distal end of the distal tube 308.

The variation comprising an endoscope 330 provides, among other things, vision at or near the distal end 306 to help assess treatment progress and determine location of the distal end 306 and a distal end of the electrode array 320 and/or electrode sections within the lumen.

In some variations, the balloon 300 is affixed to the overtube O, or to the elongate member or endoscope 330 when the overtube O is not present, such that translation and rotation of the elongate member or endoscope 330 results in a 1:1 translation and rotation of the affixed balloon 300 and the elongate member or endoscope 330.

FIG. 3D illustrates a variation wherein the balloon 300 is at least partially inflated. Two pleated regions (P1 and P2) are visible, though the balloon 300 comprises three pleats, wherein the pleated regions P1, P2 are the region along which a fold occurs during deflation, and which unfold during inflation, and including the opposing surfaces (during deflation) adjacent the folding peat and on either side of the folding pleat. Each pleated region, in some variations, may comprise a stiffening member SF that may run along an outer edge of the pleated region, that is, the region or balloon surface located between stiffening members SF and wherein the pleat, e.g., P1 is disposed approximately midway between the stiffening members SF. The pleated region thus comprises two opposing surfaces that are in at least partial contact with each other when deflated, and a pleat, e.g., P1, disposed therebetween.

In some variations, and as shown in FIG. 3D, a partial stiffening member SP may be provided along at least part of the pleat P1, P2. In some variations, the partial stiffening member SP may be located along a distal region of the pleat P1, P2. In some variations, the partial stiffening member SP may be located along a proximal region of the pleat P1, P2. In some variations, the partial stiffening member SP may be located between a proximal and distal region of the pleat P1, P2. In some variations, the partial stiffening member SP may be replaced with a stiffening member SF as described above.

Stiffening member SF and partial stiffening member SP are configured to provide a composite pleat structure that aids in inflation (unfolding) and deflation (folding) of the subject pleat as well as helping ensure that the pleats deploy, or collapse, in a desired and predetermined order.

In some variations, the stiffening member(s) SF, SP may help ensure proper pleat closure and maintain alignment between opposing magnets in each magnet pair during deflation and collapsing such that the opposing magnet pairs attract and secure the collapsed and deflated balloon.

In some variations, the stiffening member(s) SF, SP or material(s) may comprise a non-populated flex circuit material, PET, a metal such as stainless steel, a shape memory allow such as Nitinol, a shape memory polymer and/or a thin sheet or sheets, or coil(s) to stiffen portions of the pleat.

In some variations, the stiffening member(s) SF, or SP may comprise substructure of the inflatable balloon. For example, such stiffening member(s) SF, SP may be stiffening wires formed of a shape memory allow such as nitinol, a shape memory polymer, or stainless steel or PET, or the like. Such stiffening wires may run the length, or part of the length, of the balloon and be located along an outer edge of the opposing pleated regions and/or along the folding pleat. Such stiffening wires help induce folding of the pleat inward as pressure decreases during deflation.

In some variations, the stiffening member(s) SF, SP may bias the pleated region to expand.

In some variations, the stiffening member(s) SF, SP may bias the pleated region to collapse.

As shown in FIG. 3E, the balloon 300 of FIG. 3D is in a collapsed, deflated configuration.

The balloon 300 of FIGS. 3D and 3E is configured to move between a collapsed, deflated (and secured) configuration and an expanded inflated configuration.

In some variations, the distal end section 306 may be the first portion of an inflated balloon 300 to begin to deform, i.e., invert, during deflation.

A secured configuration is in some variations achieved by use of releasable fasteners located within pleated regions. As shown, the releasable fasteners comprise magnets of opposing polarity to take advantage of magnetic forces to close and open the pleated regions in a predetermined order as well as secure the closed pleated regions.

Thus, e.g., the balloon 300 may comprise an opposing polarity magnet pair M1(S), M1′(N), located on opposing pleated regions as in FIG. 3D. In some variations, and as shown in FIG. 3D, a second magnet pair M2(S), M2′(N) may be provided at a different location along the opposing pleated regions. The exemplary magnet arrangement of FIG. 3D provides a pleated region comprising the same magnetic polarity, e.g., M1(S) and M2(S). However, as the artisan will appreciate, the polarities of the magnet pairs only need to be opposing to provide the required attractive forces.

Further, the magnet designated M2′(N) also serves as part of the magnet pair M2′(N), M2″(S), wherein magnet M2″(S) is located on a surface that is adjacent to a different pleated region P2, such that magnet M2′(N) is common to the following magnet pairs: M2(S), M2′(N) to help close and secure pleat P1, and M2′(N), M2″(S) to help close and secure pleated region P2.

FIG. 3E illustrates the balloon 300 of FIG. 3D in a collapsed, secured configuration, wherein pleats 1, 2 are collapsed and folded, and secured, e.g., by magnet pair M1, M1′. FIG. 3F shows a side view of the collapsed balloon variation 300 of FIGS. 3D and 3E.

In some variations, the balloon 300 may be inserted in a collapsed configuration into a blood vessel and expanded at a target site or region. In some variations, the balloon 300 may be inserted in a collapsed configuration into a gastrointestinal tract and expanded at a target site or region, including but not limited to the esophagus, stomach, duodenum, jejunum. In some variations, the balloon may be inserted into the large intestine in a collapsed configuration and expanded at a target site or region.

One advantageous effect of the configuration of the variations of the distal end 306 effectively interrupting the large diameter distal section 304 is that it allows for distinct and clear marking of a region that has been treated, and subsequent translation of the balloon 300, either proximally or distally, to a second treatment region.

Similarly, the distal end(s) of the overtube O and/or elongate member or endoscope 330 allow in some variations for visualization of the location of the balloon 300, and in some variations of the distal end of the balloon 300 as well as the location of the distal-most electrodes within the electrode array 320.

The balloon may comprise additional shapes and configurations, including but not limited to the location of the distal tube 308 of the distal end 306.

Target tissue may be treated using the treatment devices and/or systems comprising the balloon's described herein. For example, one or more pulse waveforms may be delivered to an electrode array of a balloon to generate a pulsed or modulated electric field. Additionally or alternatively, one or more of thermal energy (e.g., heat-based ablation, cryogenic fluid), pulsed-electric field energy, ultrasonic energy (e.g., piezoelectric transducer), vapor energy, radiofrequency energy, laser energy, and mechanical energy (e.g., blade) may be applied to treat tissue. In some variations, the electrode array may have a plurality of sections arranged circumferentially about the balloon. For example, the electrode array may have two, three, four, or five sections exposed to tissue when in the expanded configuration. The operator may confirm tissue contact with a predetermined number of electrode sections and may select the corresponding electrode sections for energy delivery from a signal generator. In some variations, one or more of the electrode sections may be separated by a pleat such that an unfolded pleat will increase a distance between the electrode sections and increase a diameter of the balloon while a folded pleat will conversely decrease a distance between the electrode sections and decrease a diameter of the balloon.

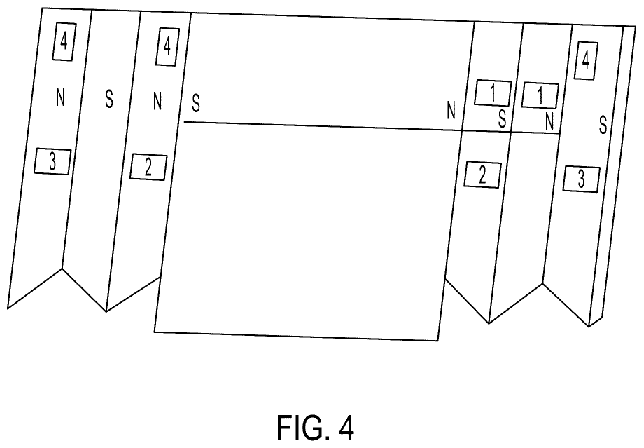

FIG. 4 illustrates one variation of a balloon such as that described above in connection with FIGS. 3A-3F. Thus, FIG. 4 illustrates balloon pleated areas through the vertical folding lines. In addition, each pleat contains one or more magnets that are configured to correspond to a magnetic polarity. The larger central region comprises, inter alia, the distal cylindrical region of FIGS. 3A-3F. The rectangles marked within the smaller pleated areas represent magnets that comprise the labeled polarity. There could be both distal and proximal pleats. In theory, the distal magnets could also be opposite polarity to the proximal pleats and therefore each pleat would not be all the same polarity. Thus, as shown, the rectangular magnets are each labeled with a number to represent exemplary magnet pairs. Therefore, 1:1, 2:2, 3:3; and 4:4:4 are magnet pairs that, when associated in close enough proximity for the magnetic force to engage between magnet pairs, the magnets are held together, thereby assisting in the folding pleating process, as well as securing the folded pleat together. There are in this exemplary variation, therefore, three magnetic pairs of 2 magnets each, and a 4^{th} and a 5^{th} pair of magnets provided by 3 magnets. As the artisan will appreciate, a wide range of possible magnetic combinations, and locations, may be achieved, each of which is within the scope of the present disclosure. In addition, in some variations, the magnetic forces between a magnetic pair may be exerted directly from magnet to magnet, without material between the magnets. In some variations, balloon material may be disposed between the magnet pairs.

In general, all balloon shapes and profiles disclosed herein may comprise electrodes or electrode sections or an electrode array, in a 360 degree surrounding configuration, or less than a 360 degree configuration, functioning to treat a condition through application of an electric field and/or inflation. Similarly, all balloon shapes and profiles discussed herein may also be provided without electrodes, functioning to treat a condition through inflation only.

The geometry, dimensions, material, and properties of the balloon may provide strength to inflate and provide apposition to tissue while also having sufficient flexibility to reproducibly be positioned circumferentially between a sheath and visualization device roll and unroll without becoming entangled with itself or tissue. In some variations, the balloon may be composed of a material biased to form a predetermined shape. For example, the balloon may comprise one or more of a flexible polymeric material (e.g., polyamide, PET), a thermoplastic urethane, thermoplastic elastomer, polyethylene terephthalate, polyimide, nylon, biaxially-oriented polyethylene terephthalate, nitinol, combinations thereof, and the like. In some variations, the balloon may comprise one or more stiffening members configured to add stiffness and/or strength in a treatment configuration.

In some variations, the size and shape of the balloon may be adjustable based on a configuration of the balloon. For example, rolling and flattening of a balloon may be caused by respective retraction and advancement of a sheath (e.g., delivery catheter) over the balloon. In some variations, the balloon and the visualization device in the delivery configuration may have a diameter of less than about 17 to about 25 mm. In some variations, the balloon in the treatment configuration may have a diameter between about 10 mm and about 90 mm, between about 20 mm and about 50 mm, between about 30 mm and about 50 mm, and between about 40 mm and about 50 mm, including all ranges and sub-values in-between.

The pleat(s) may be configured to transition the balloon (e.g., an inflatable balloon) between a folded configuration having a first diameter and an unfolded configuration having a second diameter larger than the first diameter. The second diameter may be between about 1 mm and 10 mm larger than the first diameter. For example, the balloon may be partially inflated in a folded treatment configuration where the pleat(s) remains folded but in contact with tissue. The folded configuration may be appropriate for relatively smaller tissue lumens. The balloon may be further inflated in an unfolded treatment configuration where the pleat is unfolded such that the balloon may have a larger diameter relative to the folded configuration. The unfolded configuration may be appropriate for relatively larger tissue lumens.

In some variations, the balloon may be asymmetric relative to a longitudinal axis of the balloon to facilitate a transition between a delivery configuration and treatment configuration of the balloon. For example, the asymmetry of the balloon may facilitate rolling the balloon at least partially around the visualization device when transitioning from the treatment configuration to the delivery configuration. In some variations, a length of a taper and/or an angle of the taper relative to a longitudinal axis may be different between a first taper (e.g., left side taper) and a second taper (e.g., right side taper). An asymmetric taper may allow one of the tapers to preferentially roll beneath the other taper to promote the transition of the balloon into the delivery configuration. For example, a larger taper may encounter less resistance in rolling around a visualization device. Therefore, having asymmetrical tapers may bias different portions of the balloon when withdrawing a balloon into a sheath (e.g., transitioning from a treatment configuration to a delivery configuration).

In some variations, the balloon may be asymmetrically coupled to the elongate body in order to facilitate a transition of the balloon into a delivery configuration. For example, the balloon may be eccentrically coupled to the elongate body such that a longitudinal axis of the elongate body does not align with the longitudinal axis of the balloon. In this manner, a first lateral portion of the balloon may have a smaller surface area than a second lateral portion of the balloon. In some variations, the elongate body may be coupled to a sidewall of the balloon to facilitate a transition of the balloon into a delivery configuration. For example, the elongate body may be coupled to the sidewall of the balloon such that when transitioned to the delivery configuration, a first lateral portion of the balloon may overlap a second lateral portion of the balloon when placed around a visualization device.

In other variations, the balloon may be symmetric and symmetrically coupled to the elongate body.

In some variations, the balloon may have a width of at least 10 mm, between about 10 mm and about 100 mm, between about 10 mm and about 50 mm, between about 10 mm and about 30 mm, between about 20 mm and about 40 mm, between about 30 mm and about 100 mm, between about 30 mm and about 80 mm, between about 30 mm and about 60 mm, between about 30 mm and about 50 mm, including all ranges and sub-values in-between. In some variations, the balloon may comprise a length of between about 10 mm and about 300 mm, between about 10 mm and about 200 mm, between about 10 mm and about 100 mm, between about 50 mm and about 300 mm, between about 50 mm and about 200 mm, between about 100 mm and about 300 mm, between about 100 mm and about 200 mm, including all ranges and sub-values in-between. In some variations, the balloon may comprise a wall thickness of between about 0.02 mm and about 0.5 mm, between about 0.02 mm and about 0.3 mm, between about 0.02 mm and about 0.3 mm, between about 0.02 mm and about 0.2 mm, between about 0.1 mm and about 0.5 mm, between about 0.1 mm and about 0.3 mm, between about 0.2 mm and about 0.5 mm, between about 0.3 mm and about 0.5 mm, including all ranges and sub-values in-between.

### Electrode Array

Generally, treatment members described herein may be configured to treat tissue using any of the treatment modalities described herein, including but not limited to thermal energy (e.g., heat-based ablation, cryogenic fluid), pulsed-electric field energy, ultrasonic energy (e.g., piezoelectric transducer), vapor energy, radiofrequency energy, laser energy, mechanical energy (e.g., blade), and the like. For example, the treatment member may comprise one or more of an electrode, an electrode array, a piezoelectric transducer, a laser, a blade, and a thermal element.

In some variations, the electrode array may comprise a flexible circuit substrate, wherein the flexible circuit substrate comprises one or more of the group consisting of: all-Polyimide laminate, Polyester (PET), Polyethylene Naphthalate (PEN), Polyamide, Liquid Crystal Polymer (LCP), and PTFE.

In some variations, tissue treatment characteristics may be controlled by the size, shape, spacing, composition, and/or geometry of the electrode array. For example, the electrode array may be flexible to conform to non-planar tissue surfaces. In some variations, the electrode array may be embossed or reflowed to form a non-planar electrode surface. In some variations, the electrode array may comprise a tissue contact layer. The tissue contact layer may function as a salt bridge between the electrodes and tissue. In some variations, the electrode array may comprise a hydrophilic coating. Additionally or alternatively, the electrode array may be electrically divided into sub-arrays to reduce drive current requirements. In some variations, the sub-arrays may correspond to the plurality of sections described herein.

In some variations, raised and/or rounded (e.g., semi-ellipsoid) electrodes may generally promote more reliable contact with tissue than flat electrodes and therefore a more uniform electrical field and improved treatment outcomes. For example, tissue contact (e.g., apposition) with the electrodes completes an electrical circuit during energy delivery and therefore provides the resistance in the circuit for a uniform electric field distribution. The raised and/or rounded (e.g., semi-ellipsoid) electrodes may reduce sharp edges to reduce arcing. The spaced-apart electrodes of the electrode array may further reduce ion concentration and associated electrolysis. The electrode array configurations (e.g., geometry, spacing, shape, size) shown and described herein provide uniform and spaced-apart electrodes that also allow a corresponding balloon to repeatedly expand and compress. For example, a predetermined spacing between electrodes may be maintained as a balloon upon which the electrode array is disposed increases and decreases in diameter.

In some variations, the electrode array may be connected by one or more leads (e.g., conductive wire, lead wire) to a signal generator. For example, a lead may extend through an elongate body (e.g., outer catheter, outer elongate body) to the electrode array. One or more portions of the lead may be insulated (e.g., PTFE, ETFE, ePTFE, PET, polyolefin, parylene, FEP, silicone, nylon, PEEK, polyimide, polylurethane). The lead may be configured to sustain a predetermined voltage potential without dielectric breakdown of its corresponding insulation. In some variations, the electrode array may be coupled to the balloon via a thermal seal.

In some variations, a drive voltage applied to the electrode array may depend at least on the spacing between electrodes of the electrode array as well as electrode dimensions. For example, relatively wide elongate electrodes may reduce the effect of strong electric field intensities at sharply curved edges.

Additionally or alternatively, the plurality of elongate electrodes may comprise an interdigitated configuration. For example, the plurality of elongate electrodes may comprise a curved shape (e.g., S-shape, W-shape). The electrode array may be configured to modify a flexural stiffness of the balloon to facilitate consistent expansion and compression of the balloon. In some variations, the electrode array may comprise a plurality of electrodes configured to protrude and/or recess relative to a surface of the substrate.

In some variations, a more uniform treatment of tissue (e.g., in areas where the electrode groups intersect) may be obtained by reducing the widths of the end-most electrodes of each section and reducing the distance between those electrodes. In some variations, a more uniform treatment of tissue (e.g., in areas where the electrode sections intersect) may be enabled by interdigitating the end-most electrodes of each group to overlap the treatment areas.

In some variations, an electrode array may comprise a plurality of electrode sections (e.g., zones), including 1, 2, 3, 4, 5, 6, 7, 8, 9, and 10 or more electrode sections. In some variations, each section of the plurality of sections may comprise a plurality of electrodes. For example, each section of the plurality of sections may comprises between 10 and 18 electrodes. In some variations, an electrode section of an electrode array may have a surface area of between about 250 mm² and about 1000 mm², between about 250 mm² and about 750 mm², between about 500 mm² and about 1000 mm², between 400 mm² and about 500 mm² and between 400 mm² and about 600 mm², including all ranges and sub-values in-between.

In some variations, an amount of tissue compliance may correspond to an amount of dilation and suction needed to ensure uniform surface contact of the electrodes and the desired tissue treatment. In some variations, the tissue may respond better to less dilation and more suction (or vice versa) depending on compliance and structure. In some variations, apposition may be assessed visually and/or through impedance measurement and/or through a pressure check. In some variations, apposition may be measured using one or more temperature sensors, pressure sensors, and proximity sensors. Such suction may be provided by a suction catheter or lumen that is in fluid or operative communication with the endoscope in some variations.

In some variations, one or more status checks may be issued by the system to determine status of electrode deployment. In some cases, one or more pleats may have not unfolded properly, causing the electrodes on the unfolded pleat to be shorted to itself. In some cases, the electrodes may have deployed properly, but with insufficient contact with target tissue.

Accordingly, the pulse generator may be prompted to issue a status check, or the status check may be automatically generated and issued by the pulse generator. In some variations, the generator may generate and send a single status check pulse to each electrode section after inflation of the balloon. In some variations, one or more status check pulse may be generated and sent to each electrode section. For example, less than 10 pulses and, in a preferred embodiment, less than 5 status check pulses may be generated and sent.

In some variations, the current status check may be executed by generating one or more pulses that are of therapeutic strength or magnitude. In some variations, the current status check may be executed by generating one or more sub-therapeutic pulses that are less than therapeutic strength or magnitude.

In some variations, the current status check may be executed by generating a number of pulses that equal to a therapeutic number of pulses. In some variations, the current status check may be executed by generating a number of sub-therapeutic pulses that are less than a therapeutic number of pulses.

In some variations, the current status check may comprise any combination of the following: generating one or more pulses that are of therapeutic strength or magnitude; generating a number of pulses that equal to a therapeutic number of pulses; and/or generating a number of sub-therapeutic pulses that are less than a therapeutic number of pulses.

In some variations, the current status check may comprise any combination of the following: generating one or more pulses that are less than therapeutic strength or magnitude; generating a number of pulses that equal to a therapeutic number of pulses; and/or generating a number of sub-therapeutic pulses that are less than a therapeutic number of pulses.

The pulse generator is configured to measure the status check current value(s) that pass through the electrodes and return to the generator. The pulse generator, or controller operatively attached to the pulse generator, is configured to determine if the status check current value or magnitude is within predetermined limits, e.g., below an upper predetermined limit and/or above a predetermined lower limit. If the status check current value is within the predetermined limit(s), the controller or pulse generator allow pulses to be issued to that specific electrode section. If the status check current value for a particular electrode section is outside the predetermined limit(s), may indicate that the electrode section did not properly deploy and/or that the electrode section is not in sufficient contact with the target tissue. In some variations, the operator may be notified by annunciation via the pulse generator or controller of the results of the status current check for each electrode section. In some variations, an out-of-compliance status check current value may be corrected by the operator deflating the balloon at least partially and attempting to reinflate and executing another current status check. In some variations, the operator may deflate the balloon, then translate and/or rotate the balloon slightly, then reinflate the balloon within the target region and execute another current status check. In some variations, the pulse generator and/or controller may be configured to not allow pulses to be issued to the out-of-compliance electrode section, or the out-of-compliance electrode section may be deactivated.

An alternative status check for proper electrode section deployment may comprise an impedance check in order to determine whether the electrodes have properly deployed and are in sufficient contact with tissue.

Further, the controller and/or pulse generator may be configured to execute a balloon pressure measurement to determine if the balloon is sufficiently pressured to ensure proper tissue apposition that is sufficient to execute a therapeutic series of pulses, or to conduct an electrode status check as discussed above.

Moreover, variations of the systems described herein may monitor voltage and/or current during execution of pulsed energy generation and application to target tissue. The controller and/or pulse generator may comprise predetermined threshold limits for current and/or voltage. If, in some variations, current and/or voltage is measured at a magnitude that is outside the predetermined upper and/or lower limits, the controller and/or pulse generator is configured to stop generating voltage pulses.

In some variations, the systems described herein may be configured to stop generating voltage pulses that occur within a programmed series of bursts of pulses. For example, a current and/or voltage may be measured that is outside the predetermined upper and/or lower limits. When this occurs, the controller and/or generator stop generating voltage pulses. This stop-generating instruction may be issued in the midst of a programmed 2^{nd} burst of pulses that is part of a larger number of pulse bursts, e.g., 5 or more bursts of pulses may be programmed. When the voltage pulse stop generation instruction is issued during a burst of pulses that is part of a larger programmed number of serial pulse bursts, in some variations, the controller or pulse generator issue an instruction to abort the current burst of pulses and return to initiate the first pulse in that current burst of pulses. Stated differently, the controller or pulse generator may not pause the burst of pulses, then resume where the stop-voltage pulse instructions were issued. Instead, when the burst of pulses is interrupted, the controller or pulse generator instruct restarting the interrupted burst of pulses at pulse number 1 of burst number 1 of a series of bursts. Alternatively, the instructions may restart the burst of pulses at pulse number 1 of the burst that was interrupted.

In addition, as discussed supra, the magnet pairs may be arranged in a predetermined order of magnetic force strength to ensure a predetermined opening and closing of the pleats.

In some variations, an elastic band may be provided to aid in securing the balloon in a deployed or collapsed configuration. When the balloon is rolled up or folded, the elastic band can be attached to the cuff and stretched over the folded balloon to hold it in the deployment position. Then when the balloon is inflated, the elastic band may roll off distally onto the cuff and remains there for the remainder of the procedure, out of the way of the balloon and electrodes. This is beneficial for getting the device into a delivery configuration to make insertion easier especially with the circular distal end that does not have a tapered transition. This would not be needed on removal of the device as the scope is leading the way and the proximal end of the balloon is in some variations tapered and therefore will move on its own to accommodate the sphincters that need to be traversed. As discussed, this could also be accomplished by putting a thin band that is not attached that actually rolls proximally and ends up over the scope so its not entirely detached. Additionally, this could be accomplished through water soluble adhesive or tape to keep it in the delivery configuration until it degrades in the body and the balloon inflation tears it. In further embodiments, the balloon may be heat set in a delivery or collapsed configuration in which the heat setting process is able to substantially maintain the delivery configuration until inflation or expansion of the balloon takes place. For example, to heat set the balloon it may be constrained in a delivery or collapsed configuration and exposed to 50-75 degrees Celsius for 5 min to 12 hrs.

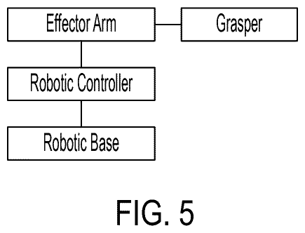

FIG. 5 illustrates one illustrative variation of a system according to the present disclosure driven at least in part by a robotic system. Therefore, in some variations, a treatment executed by a treatment device comprising an elongate member, or an endoscope with balloon affixed thereto, is executed by a robotic system. In some variations, the robotic system comprises a robotic controller having a memory, processor and programmed instructions configured to instruct the robotic system to execute the treatment. In some variations, the robotic system comprises a robotic base and an effector arm comprising a grasper configured to grasp the elongate member or endoscope. In some variations, the robotic system is configured to inflate and deflate the balloon. In some variations, the robotic system is configured to automatically inflate the balloon at a first treatment region, execute a first treatment, at least partially deflate the balloon, translate the balloon to a second treatment region, wherein the second treatment region overlaps the first treatment region, inflate the balloon and execute a second treatment. In some variations, the robotic system is configured to automatically execute a status check to determine proper balloon deployment and/or sufficient electrode contact with tissue as described supra. In some variations, one or more of these steps may be executed manually.

It should be understood that the examples and illustrations in this disclosure serve exemplary purposes and departures and variations such as the number of electrodes and devices, and so on can be built and deployed according to the teachings herein without departing from the scope of this invention.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

It should be understood that the examples and illustrations in this disclosure serve exemplary purposes and departures and variations such as the number of electrodes and devices, and so on can be built and deployed according to the teachings herein without departing from the scope of this invention.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

The specific examples and descriptions herein are exemplary in nature and variations may be developed by those skilled in the art based on the material taught herein without departing from the scope of the present invention, which is limited only by the attached claims.

1. An intralumenal treatment device, comprising:
an elongate body;
an inflatable balloon eccentrically surrounding the elongate body, wherein the inflatable balloon is asymmetric relative to a longitudinal axis through the elongate body;
wherein the inflatable balloon comprises
a proximal section;
a cylindrical distal section adjacent and distal to the proximal section,
wherein the cylindrical distal section comprises a circular distal end having a diameter that is the same as a diameter of the cylindrical distal section,
wherein the circular distal end defines a tubular aperture therethrough that is in fluid communication with an interior of the inflatable balloon, and
wherein the tubular aperture is configured to receive and couple to the elongate body;
a plurality of pleats, each pleat comprising first and second opposing pleated regions associated with, and adjacent to, the pleat.

2. The intralumenal treatment device of claim 1, wherein each of the first and second opposing pleated regions associated with each pleat in the plurality of pleats comprises a releasable fastener configured to releasably secure each pleat in the plurality of pleats in a folded, deflated and secured configuration, and
wherein, during an inflation of the inflatable balloon that generates sufficient inflation pressure to release each releasable fastener, each pleat in the plurality of pleats, and each of the associated opposing pleated regions, are configured to transition to an at least partially inflated and expanded configuration in a predetermined order.

3. The intralumenal treatment device of claim 1, wherein the elongate body comprises an endoscope.

4. The intralumenal treatment device of claim 3, further comprising an overtube surrounding at least a distal region of the elongate body, wherein the inflatable balloon surrounds and is affixed to the overtube.

5. The intralumenal treatment device of claim 1, wherein the cylindrical distal section comprises an array of electrodes coupled to the cylindrical distal section.

6. The intralumenal treatment device of claim 1, wherein the circular distal end of the distal section is substantially flat.

7. The intralumenal treatment device of claim 6, wherein the circular distal end of the distal section is substantially transverse to a longitudinal axis of the elongate body when the inflatable balloon is in an inflated configuration.

8. The intralumenal treatment device of claim 1, wherein a portion of the circular distal end of the distal section is everted away from the cylindrical distal section when the inflatable balloon is in an inflated configuration.

9. The intralumenal treatment device of claim 1, wherein a portion of the circular distal end of the distal section is inverted into the cylindrical distal section when the inflatable balloon is in an deflated configuration.

10. The intralumenal treatment device of claim 1, wherein a first pleat in the plurality of pleats, and the first and second opposing pleated regions associated with the first pleat, is configured to transition from the closed and deflated configuration to the at least partially inflated and expanded configuration before a second pleat in the plurality of pleats, and the first and second opposing pleated regions associated with the second pleat, transitions from the closed, deflated and secured configuration to an at least partially inflated and expanded configuration.

11. The intralumenal treatment device of claim 10, wherein an inflation pressure sufficient to transition the second pleat, and the first and second opposing pleated regions associated with the second pleat, to the at least partially inflated and expanded configuration is less than an inflation pressure sufficient to transition the first pleat and the first and second opposing pleated regions associated with the first pleat, to the at least partially inflated and expanded configuration.

12. The intralumenal treatment device of claim 1, wherein the second pleat, and the first and second opposing pleated regions associated with the second pleat, are configured to transition from the closed and deflated configuration to the at least partially inflated and expanded configuration at substantially the same time as the first pleat, and the first and second opposing pleated regions associated with the first pleat, transitions from the closed, deflated and secured configuration to an at least partially inflated and expanded configuration.

13. The intralumenal treatment device of claim 12, wherein an inflation pressure sufficient to transition the second pleat, and the first and second opposing pleated regions associated with the second pleat, to the at least partially inflated and expanded configuration is substantially the same as an inflation pressure sufficient to transition the first pleat, and the first and second opposing pleated regions associated with the first pleat, to the at least partially inflated and expanded configuration.

14. The intralumenal device of claim 1, wherein the inflatable balloon comprises two pleats.

15. The intralumenal treatment device of claim 1, wherein the releasable fastener comprises at least one of the group consisting of: opposing attractive magnets, a hook, loop or pin system, biocompatible sutures or fibers or tether configured to break at a designated force or pressure, an adhesive, a heat-sensitive polymer with controlled degradation at body temperature, a shape-memory alloy such as nitinol, and shape-memory polymers.

16. The intralumenal treatment device of claim 1, wherein each of the first and second releasable fasteners comprises a pair of opposing magnets configured to attract each other, wherein a first pair of opposing magnets is associated with a first pleat in the plurality of pleats, wherein a first magnet in the first pair of opposing magnets is located on a first opposing pleated region associated with the first pleat and a second magnet in the first pair of opposing magnets is located on a second opposing pleated region associated with the first pleat, and wherein the first pair opposing magnets are configured to exert an attraction force comprising a maximum magnitude therebetween when the opposing magnets are in engaged proximity with each other to releasably seal the opposing pleated regions together.

17. The intralumenal treatment device of claim 16, wherein when the generated inflation pressure is greater than the attraction force's maximum magnitude, the first pair of opposing magnets are configured to disengage and allow the first pleat to unfold, and the first and second opposing pleated regions to move apart from each other.

18. The intralumenal treatment device of claim 16, wherein when the opposing magnets move apart from each other, the first pleat, and the first and second opposing pleated regions associated with the first pleat, are configured to move to an at least partially inflated configuration.

19. The intralumenal treatment device of claim 18, wherein deflation of the first pleat, and the first and second opposing pleated regions associated with the first pleat, is configured to reengage the pair of opposing magnets such that the attractive force between the opposing magnets is at a maximum magnitude.

20. The intralumenal treatment device of claim 1, wherein the inflatable balloon comprises a stiffening member at an edge of each of the first and second pleated regions adjacent to the first and second pleats.

21. The intralumenal treatment device of claim 1, wherein the inflatable balloon comprises a partial stiffening member located along a partial length of an edge of each of the first pleat and the second pleat.

22. The intralumenal treatment device of claim 1, wherein the proximal section of the balloon is one of tapered or circular.

23. An inflatable intralumenal balloon, comprising:
a proximal section;
a cylindrical distal section adjacent and distal to the proximal section,
wherein the cylindrical distal section comprises a circular distal end having a diameter that is the same as a diameter of the cylindrical distal section,
wherein the circular distal end defines a tubular aperture therethrough that is in fluid communication with an interior of the inflatable balloon, and
wherein the tubular aperture is configured to receive and couple to the elongate body.

24. The inflatable intralumenal balloon of claim 23, wherein the proximal section of the balloon is one of tapered or non-tapered.

25. The inflatable intralumenal balloon of claim 23, further comprising a plurality of pleats, each pleat comprising first and second opposing pleated regions associated with, and adjacent to, the pleat.

26. The inflatable intralumenal balloon of claim 25 wherein each of the first and second opposing pleated regions associated with each pleat in the plurality of pleats comprises a releasable fastener configured to releasably secure each pleat in the plurality of pleats in a folded, deflated and secured configuration.

27. The inflatable intralumenal balloon of claim 23, further comprising an array of electrodes coupled to the cylindrical distal section.

28. An inflatable intralumenal balloon, comprising:
a proximal section, wherein the proximal section is tapered, or non-tapered;
a cylindrical distal section adjacent and distal to the proximal tapered section,
wherein the cylindrical distal section comprises a circular distal end having a diameter that is the same as a diameter of the cylindrical distal section, and
wherein the circular distal end defines a tubular aperture therethrough that is in fluid communication with an interior of the inflatable balloon.

29. The inflatable intralumenal balloon of claim 28, comprising an array of electrodes coupled to the cylindrical distal section.

30. The inflatable intralumenal balloon of claim 28, wherein the proximal section is non-tapered and circular.

31. The inflatable intralumenal balloon of claim 30, wherein the tubular aperture is not centered within the circular distal end.

32. A system comprising the intralumenal treatment device of claim 1, further comprising a pulse generator and/or controller, wherein the pulse generator is in operative electrical communication with an electrode array comprising a plurality of electrode sections and coupled to the cylindrical distal section, the pulse generator configured to generate electrical pulses to the electrode array.

33. The system of claim 32, wherein the pulse generator or the controller comprise instructions configured to automatically detect which electrode sections to send the generated electrical pulses.

34. The system of claim 33, wherein the pulse generator is configured to send at least one pulse of non-therapeutic voltage and current magnitude, measure the current that passes through each of the electrode sections, and determining if the measured current magnitude is within predetermined limits.

35. The system of claim 34, wherein if the measured current magnitude of an electrode section is outside of the predetermined limits, then the pulse generator is configured to not send therapeutic pulses to that electrode section.

36. The system of claim 32, wherein the pulse generator and/or controller are programmed to generate a plurality of bursts of voltage pulses.

37. The system of claim 36, wherein the pulse generator and/or controller are configured to monitor the voltage and/or current of each pulse within a burst of voltage pulses and compare against predetermined limits.

38. The system of claim 37, wherein if the monitored voltage and/or current is outside the predetermined limits, the pulse generator and/or controller are configured to interrupt the burst of pulses.

39. The system of claim 38, wherein the pulse generator and/or controller are configured to restart the interrupted burst of pulses at a first pulse in the burst of pulses such that the entire interrupted burst of pulses is initiated and repeated in full.

40. The system of claim 39, further comprising a robotic system comprising:
a robotic base and a drive unit associated with the robotic base;
a robotic arm in operative connection with the robotic base;
an effector arm operatively connected with the robotic arm and configured to move in more than one dimension;
a grasper operatively connected with the effector arm and configured to grasp and manipulate a proximal end of the elongate member and/or endoscope;
and a robotic controller configured to control and manipulate the effector arm and the grasper according to user inputs and/or programmed instructions;
wherein the robotic system is configured to perform at least some of the following:
translate the inflatable balloon in a collapsed configuration through the bodily lumen to a target region comprising a first target tissue;
inflate the inflatable balloon such that the electrodes contact a first target tissue;
generate at least one voltage pulse with the pulse generator and energize the electrode array to treat the first target tissue;
at least partially deflate the inflatable balloon;
translate the inflatable balloon to a second target tissue that comprises an overlap with the first target tissue;
reinflate the inflatable balloon such that the electrode contact the target tissue; and
generate at least one voltage pulse with the pulse generator and energize the electrode array to treat the target tissue.

41. A method of delivering energy pulses to a bodily lumen, comprising:
providing a system according to claim 38, and comprising a pulse generator in electrical communication with an electrode array that is coupled with the cylindrical distal section of the inflatable balloon;
translating the inflatable balloon in a collapsed configuration through the bodily lumen to a target region comprising a first target tissue;
inflating the inflatable balloon such that the electrodes contact the first target tissue;
generating at least one voltage pulse with the pulse generator and energizing the electrode array to treat the first target tissue;
at least partially deflating the inflatable balloon;
translating the inflatable balloon to a second target tissue that comprises an overlap with the first target tissue;
reinflating the inflatable balloon such that the electrode contact the target tissue; and
generating at least one voltage pulse with the pulse generator and energizing the electrode array to treat the target tissue.

42. The method of claim 41, wherein the overlap of the second target tissue and the first target tissue results in treating the overlapped tissue twice.

43. The method of claim 41, further comprising before generating the at least one voltage pulse, confirming that the electrodes are in sufficient contact with the target tissue.

44. The method of claim 43, wherein confirming that the electrodes are in sufficient contact with the target tissue comprises one or more of an impedance check, a current check, and/or a balloon pressure check.

45. The method of claim 41, wherein the inflatable balloon is configured to be translated and/or rotated in a 1:1 relationship with an endoscope.
